(19) United States
(12) Patent Application Publication
(10) Pub. No.: US 2005/0065429 A1
(43) Pub. Date: Mar. 24, 2005

(54) Method for three plane interleaved acquisition for three dimensional temperature monitoring with MRI

(75) Inventor: Yong Zhou, Waukesha, WI (US)

Correspondence Address: JOSEPH S. HEINO, ESQ., 111 E. KILBOURN AVENUE, SUITE 1400, MILWAUKEE, WI, 53202, US

(73) Assignee: GE Medical Systems Global Technology Company, LLC, Waukesha, WI (US)

(21) Appl. No.: 10/666,656

(22) Filed: Sep. 18, 2003

(51) Int. Cl. A61B 5/05

(52) U.S. Cl. 600/412

(57) ABSTRACT

The present invention provides for a method of three dimensional temperature monitoring based in hyperthermic procedures, the heat dissipation, and therefore, temperature variations, does not change dramatically over a spatial region and, second, temperature change, which manifests itself as the phase change, has different characteristics compared to the anatomic magnitude image. Therefore the present invention exploits these facts to provide a method for acquiring the temperature map of three orthogonal planes nearly simultaneously.

### BACKGROUND OF THE INVENTION

The present invention relates generally to a magnetic resonance imaging (MRI) methods and devices, and more specifically, to a method for using MRI to measure temperature change in either liquid or tissue.

Thermal energy deposition is often used in medicine as a means of necrosing diseased tissues. Lasers, radio frequency antennas and ultrasonic transducers are examples of devices used for the deposition of thermal energy for therapy. However, regardless of the therapeutic regimen used, it is desirable to have a means of guiding and monitoring this energy deposition to assure the energy is applied in the proper location and to verify that appropriate energy levels are used to prevent undertreatment or overtreatment for two main reasons. The first is to ensure that the diseased tissue has been exposed to an adequate temperature-time treatment to induce necrosis over the entire diseased volume. The second is to ensure that the surrounding healthy tissue is spared excess thermal treatment. Magnetic resonance imaging has been demonstrated as a method for identifying regions of tissue to be treated, guiding therapeutic devices and monitoring the deposition of thermal energy from lasers, ultrasound devices or cryogenic probes.

When a substance such as human tissue is subjected to a uniform magnetic field (polarizing field B₀), the individual magnetic moments of the spins in the tissue attempt to align with this polarizing field, but precess about it in random order at their characteristic Larmor frequency. If the substance, or tissue, is subjected to a magnetic field (excitation field B₁) which is in the X-Y plane and which is near the Larmor frequency, the net aligned moment, M_{z}, may be rotated, or “tipped”, into the X-Y plane to produce a net transverse magnetic moment Mₜ. A nuclear magnetic resonance (NMR) signal is emitted by the excited spins after the excitation signal B₁ is terminated and this signal may be received and processed to form an image.

When utilizing NMR signals to produce images, a technique is employed to obtain NMR signals from specific locations in the subject. Typically, the region which is to be imaged is scanned by a sequence of NMR measurement cycles which vary according to the particular localization method being used. The region of interest may be a small portion of the patient's anatomy, such as the head or heart, or a much larger portion such as the entire thorax or spine. The resulting set of received NMR signals are digitized and processed to reconstruct the image using one of many well-known reconstruction techniques. To perform such a scan, it is necessary to elicit NMR signals from specific locations in the subject. This is accomplished by employing magnetic fields (Gₓ, G_{y} and G_{z}) which have the same direction as the polarizing field B₀, but which have a gradient along the respective X, Y and Z axes. The magnetic field gradients are produced by a trio of coil assemblies placed around the object being imaged. By controlling the strength of these gradients during each NMR measurement cycle, the spatial distribution of spin excitation can be controlled and the location of the resulting NMR signals can be identified.

MRI has many advantages compared to other imaging modalities such as computed tomography (CT), ultrasound and x-ray. One of these advantages is that MRI can be used to measure temperature change in either liquid or tissue. This allows continuous monitoring of heat dissipation during a hyperthermic procedure such as ablations using various devices. To the inventor's knowledge, no other major imaging modality has that capability.

In recent years, there has been rapid development both technically and clinically in the field of thermal imaging with MRI. In general, these techniques are employed to guide non-invasive and minimally invasive interventional procedures. It is expected that this growth trend will only continue with the improvement of the efficacy of these techniques and resulting wider acceptance of these new techniques in the clinical arena. A variety of methods are used to heat bodily tissue during these procedures. However, the most commonly used heating procedures the inventor is aware of include: laser radiation; microwave radiation; radio frequency radiation; and focused ultrasound.

Several techniques have been used to measure the temperature change including measuring temperature induced change in the longitudinal relaxation time, in the diffusion coefficient, or in the water proton resonance frequency (PRF) shift. The most robust and commonly used technique thus far is based on the PRF shift because of the linearity of the phase change with respect to the temperature change and the near independence of the tissue type.

MR temperature mapping technique based on PRF was first proposed by Ishihara et al. The Larmor precession frequency depends on the local magnetic field
ω=γB_{loc}
in which B_{loc} is determined by
B_{loc}=B₀[1+σ₀+σ(T)]
and γ is the proton gyromagnetic ratio (42.58×10⁶ Hz/T). The chemical shift terms σ₀ and σ(T) represent the temperature independent and the temperature dependent contributions respectively and are measure in ppm. The temperature independent term include the effect from B₀ field inhomogeneities. The temperature dependent term is linearly proportional to the temperature within the temperature range that is of interest to most hyperthermic treatment (40-70° C.):
σ(T)=αT
The term α represents the temperature dependence of the water proton resonance frequency and is approximately 0.01 ppm/° C. The change in temperature will cause change in the resonance frequency which in turn leads to change in the phase (Δφ) of the signal:
Δφ=φ−φ_{ref}=(ω−ω_{ref})T_{E}=α(T−T_{ref})T_{E}γB₀
in which T_{ref} is the reference temperature, normally taken at the start of the procedure, and T_{E} is the echo time. The temperature can be obtained using:

$$T = {T}_{ref} + \frac{Δφ}{α {T}_{E} γ {B}_{0}}$$

In the past, most temperature mapping has been done in a two dimensional plane with a single slice scan in order to have sufficient spatial and temporal resolution. Even though most of the heat delivery technique causes focused heating at a single spot, the subsequent heat dissipation occurs in the tissues surrounding the heated area and is intrinsically a three dimensional process. The heating pattern in the tissues may or may not be isotropic depending upon tissue composition, blood flow, and diffusion in the tissue. Therefore it is desirable to monitor the heat distribution, i.e. the temperature map in a three dimensional fashion to avoid injury to healthy tissues. However, a regular three dimensional acquisition is time consuming, so the spatial resolution or the temporal resolution will be compromised. In addition, it is not easy to present three dimensional phase information effectively due to effects of phase wraparound and noise.

### SUMMARY OF THE INVENTION

Accordingly, what is needed is a method that utilizes an MR imaging system for effectively monitoring the temperature in three dimensions. The present invention provides for such a method of three dimensional temperature monitoring. It has the advantages of being highly efficient in acquisition and being straightforward in presentation. The present invention is based on two general observations. First, in heterogeneous tissues where the hyperthermic procedure is being conducted, the heat dissipation, and therefore, temperature variations, do not change dramatically over a spatial region. Second, temperature change, which manifests itself as the phase change, has different characteristics compared to the anatomic magnitude image.

In general, methods of heating tissue from a point source, which include laser ablation, focused ultrasound ablation or RF ablation, cause a temperature profile in the tissue wherein the temperature drops gradually from the center while heat is being applied. Therefore the present invention provides a method for acquiring the temperature map of three orthogonal planes nearly simultaneously. Obviously, this will aid the clinician in regulating the hyperthermic process to target the diseased tissue and avoid damaging healthy tissue. The foregoing and other features and advantages of the present invention will be apparent from the description that follows.

### BRIEF DESCRIPTION OF THE DRAWINGS

FIG. 1 is a block diagram of an MRI system used in the present invention.

FIG. 2 is an electrical block diagram of the transceiver that forms part of the MRI system of FIG. 1.

FIG. 3 is a graphical representation of the preferred pulse sequence used to acquire the phase image data according to the present invention.

FIG. 4 is a schematic view of the phantom placement of the three orthogonal planes used in experimentation and validation of the present invention.

FIG. 5 is a pictorial view of the magnitude and phase difference images of the phantoms shown in FIG. 4.

FIG. 6 is a graph showing the temperature change recorded on three different planes.

### DETAILED DESCRIPTION

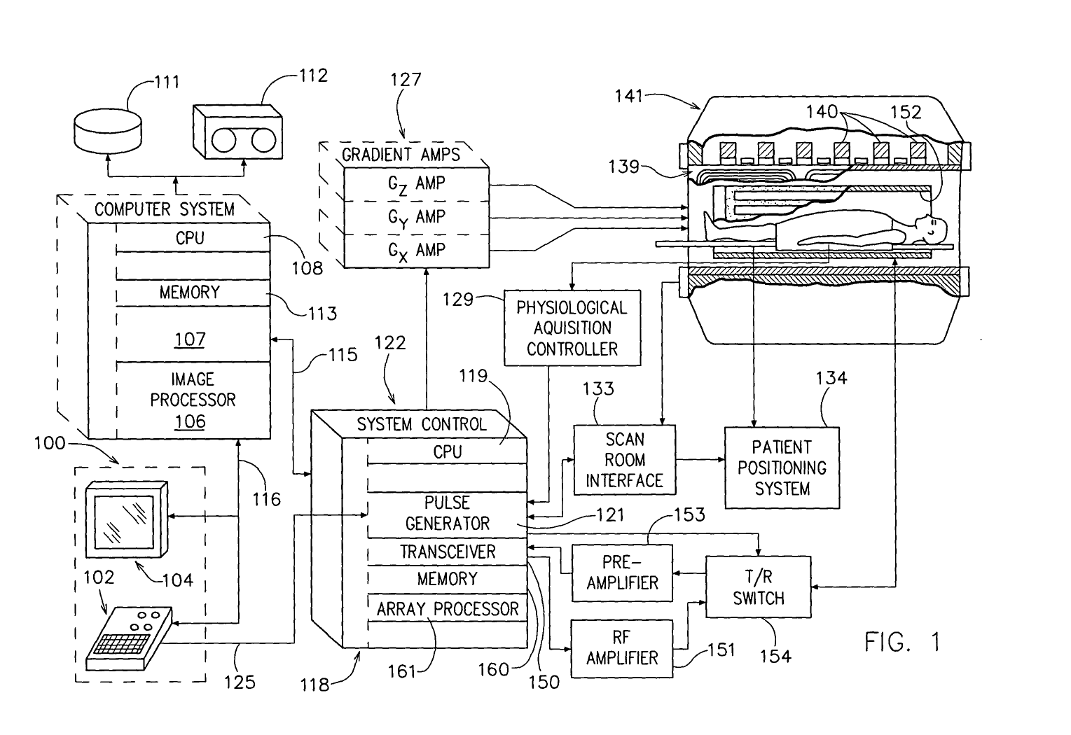

Referring now to the drawings in detail wherein like numbered elements refer to like elements throughout, FIG. 1 is a block diagram showing the components of an MRI system. The operation of the system is controlled by a console 100 which includes a keyboard, a control panel 102 and a display. The console 100 communicates through a link 116 with a separate computer system 107 that enables an operator to control the production and display of images on the screen 104. The computer system 107 includes a number of modules which communicate with each other through a backplane 118. These include an image processor module 106, a CPU module 108 and a memory module 113, known in the art as a frame buffer for storing image data arrays. The computer system 107 is linked to a disk storage 111 and a tape drive 112 through a high speed serial link 115.

The system control 122 includes a set of modules connected together by a backplane. These include a CPU module 119 and a pulse generator module 121 which connects to operator console 100 through a serial link 125. It is through this link 125 that the system control 122 receives commands from the operator which indicated the scan sequence to be performed. The pulse generator module 121 connects to a set of gradient amplifiers 127, to indicate the timing and shape of the gradient pulses to be produced during the scan. The pulse generator module 121 also receives patient data from a physiological acquisition controller 129 that receives signals from several different sensors connected to the patient, such as ECG signals from electrodes or respiratory signals from bellows. And finally, the pulse generator module 121 connects to a scan room interface circuit 133 that a patient positioning system 134 receives commands to move the patient to the desired position for the scan.

The gradient waveforms produced by the pulse generator module 121 are applied to a gradient amplifier system 127 comprised of Gₓ, G_{y} and G_{z} amplifiers. Each gradient amplifier excites a corresponding gradient coil in an assembly generally designed 139 to produce a magnetic field gradients used for position encoding acquired signals. The gradient coil assembly 139 forms part of a magnet assembly 141 which includes a polarizing magnet 140 and a whole body RF coil 152. A transceiver module 150 in the system control 122 produces pulses which are amplified by an RF amplifier 151 and coupled to the RF coil 152 by a transmit/receive switch 154. The resulting signals radiated by the excited nuclei in the patient may be sensed by the same RF coil 152 and coupled through the transmit/receive switch 154 to a preamplifier 153. The amplified NMR signals are demodulated, filtered, and digitized in the receiver section of the transceiver 150. The transmit/receive switch 154 is controlled by a signal from the pulse generator module 121 to electrically connect the RF amplifier 151 to the coil 152 during the transmit mode and to connect the preamplifier 153 during the receive mode. The transmit/receive switch also enables a separate RF coil (for example, a head coil or surface coil) to be used in either the transmit or receive mode.

The NMR signals picked up by the RF coil 152 are digitized by the transceiver module 150 and transferred to a memory module 150 in the system control 122. When the scan is completed and an entire array of data has been acquired in the memory module 160 in the system control 122, an array processor 161 operates to Fourier transform the data into an array of image data. This image data is conveyed through the serial link 115 to the computer system 107 where it is stored in the disk memory 111. In response to commands received from the operator console 100, this image data may be archived on the tape drive 112, or it may be further processed by the image process or 106 and conveyed to the operator console 100 and presented on the display 104.

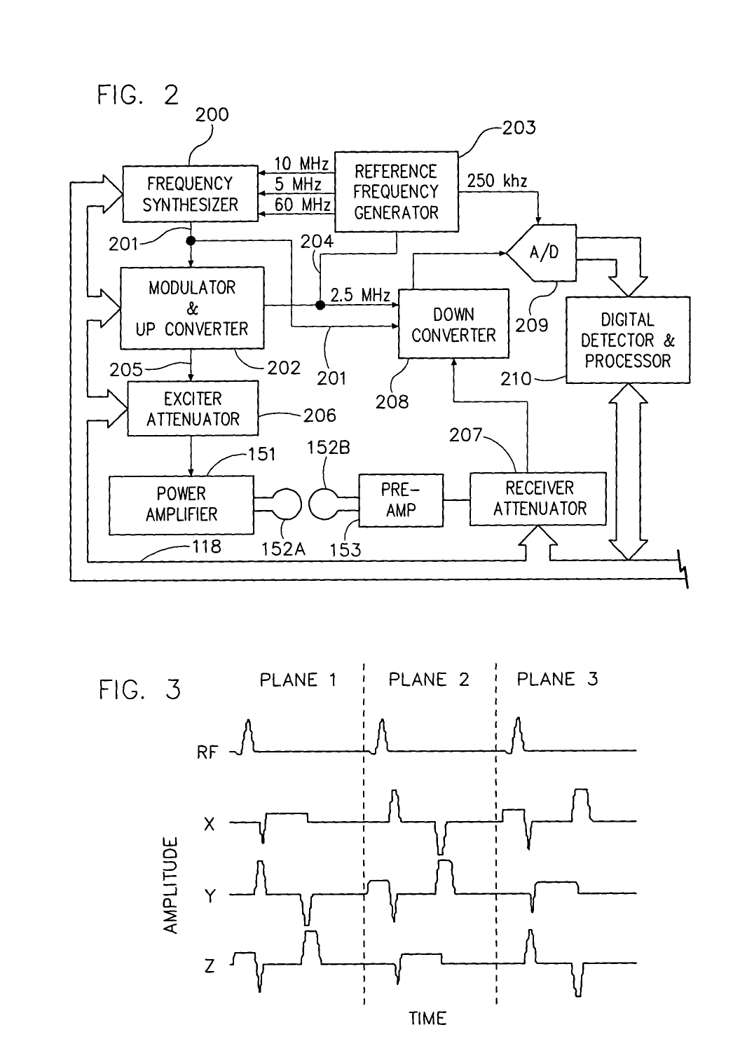

Referring particularly to FIGS. 1 and 2, the transceiver 150 produces the RF excitation field B₁ through power amplifier 151 at a coil 152A and receives the resulting signal induced in coil 152B. As indicated in FIGS. 1 and 2, coils 152A and 152B may be separate as shown in FIG. 2 and/or together as shown in FIG. 1. The base, or carrier, frequency of the RF excitation field is produced under control of a frequency synthesizer 200 which receives a set of digital signals (CF) from the CPU module 119 and pulse generator module 121. These digital signals indicate the frequency and phase of the RF carrier signal produced at an output 201. The commanded RF carrier is applied to a modulator and up converter 202 where its amplitude is modulated in response to a signal R(t) also received from the pulse generator module 121. The signal R(t) defines the envelope of the RF excitation pulse to be produced and is produced in the module 121 by sequentially reading out a series of stored digital values. These stored digital values may, in turn, be changed from the operator console 100 to enable any desired RF pulse envelope to be produced.

The magnitude of the RF excitation pulse produced at output 205 is attenuated by an exciter attenuator circuit 206 which receives a digital command, TA, from the backplane 118. The attenuated RF excitation pulses are applied to the power amplifier 151 that drives the RF coil 152A.

Referring still to FIGS. 1 and 2 the signal produced by the subject is picked up by the receiver coil 152B and applied through the preamplifier 153 to the input of a receiver attenuator 207. The receiver attenuator 207 further amplifies the signal by an amount determined by a digital attenuation signal (RA) received from the backplane 118.

The received signal is at or around the Larmor frequency, and this high frequency signal is down converted in a two step process by a down converter 208 which first mixes the NMR signal with the carrier signal on line 201 and then mixes the resulting difference signal with the 2.5 MHz reference signal on line 204. The down converted NMR signal is applied to the input of an analog-to-digital (A/D) converter 209 which samples and digitizes the analog signal and applies it to a digital detector and signal processor 210 which produces 16-bit in-phase (I) values and 16-bit quadrature (Q) values corresponding to the received signal. The resulting stream of digitized I and Q values of the received signal are output through backplane 118 to the memory module 160 where they are employed to reconstruct an image. The reference frequency generator 203 provides a reference phase for received NMR signals.

The method of the present invention employs the foregoing imaging reconstruction method and apparatus to accomplish MR temperature mapping on the basis of Water Proton Resonance Frequency (PRF) measurement which is well summarized in Quesson, B., Zwart J. A., Moonen, C. T. W., Magnetic Resonance temperature imaging for guidance of thermotherapy. J Megn Reson Imaging 2000; 12:525-533. In PRF, the local magnetic field B_{loc}(\vec{r}) as observed by the spins is a function of the main magnetic field B₀ and the chemical shift o(T(\vec{r})):
B_{loc}(T)=[1+o(T)]B₀
The chemical shift field (in ppm) is the sum of temperature-independent contributions, for example, those originating from B₀ field inhomogeneities, represented by σ₀, and a temperature-dependent contribution σ_{T}(T):
σ(T)=σ₀+σ_{T}(T)
The chemical shift field can be calculated from the phase information in RF-spoiled gradient-echo images:
Φ(T)=λσ(T)T_{E}B₀
where Φ is the image phase, λ is the gyromagnetic ratio of the observed nucleus (42.58×10⁶ Hz/T for protons), and T_{E} is echo time. To measure temperature-dependent changes in chemical shift, the term σ₀(\vec{r}) must be eliminated, which is typically accomplished by subtraction of the field distribution measured at a given reference temperature from the field distribution measured at temperature T, leading to:

$$Δ T = T - {T}_{ref} = \frac{Φ ( T ) - Φ ( {T}_{ref} )}{αγ {T}_{E} {B}_{0}}$$

where α is the temperature-dependent water chemical shift in ppm/° C. In principle, any gradient-echo method can be used for PRF-based MR thermometry, so long as contributions from simulated echoes can be neglected. RF spoiling of fast gradient echoes is thus necessary when flip angles close to the Ernst angle are used for optimal signal to noise ration (SNR) for short T(r).

The method of the present invention is based on the observation that, even in heterogeneous tissues, heat dissipation does not changed dramatically over a spatial region. Therefore, temperature variation does not change dramatically over a spatial region. Unfortunately, the temperature variation does vary as to direction. The differences in temperature variation in regards to direction may depend on the orientation of the heating device as well as the composition of the tissue being heated.

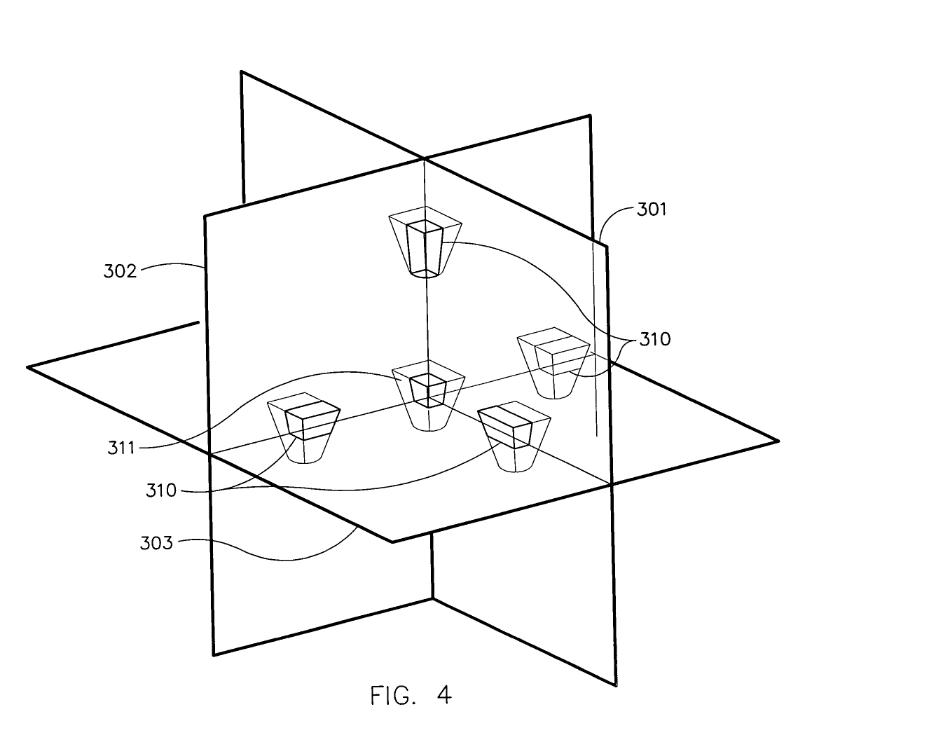

However, it is not absolutely necessary to get the temperature distribution of the entire imaging volume. It would instead be more useful to provide a temperature map of three orthogonal planes, as shown in FIG. 4. There are three basic ways of accomplishing this. First, one can use regular multi-phase scan in which one plane is acquired after another and the images of three planes are acquired in a cyclic fashion. The drawback of this technique is that the temperature maps represented by three planes are not truly simultaneous because they are not acquired simultaneously. Second, one can also partition the k-space in each imaging plane into small regions and update one region on one plane at a time. In general, k-space is a device for mathematically defining the imaging volume. Each individual point in the image is reconstructed from every point in the k-space representation of the image. For example, if 256×256 in matrix size, then the k-space is also 256×256. The images are reconstructed by combining the segmented k-space data for each plane. This method provides data that is close to simultaneous or at least better than that of the first method. One drawback to this method is that dummy pulses (disdaqs) are needed before the acquisition of each region in order to avoid image artifacts. This increases acquisition time. Lastly, and as provided for in the method of the present invention, every k-space line in one plane is acquired after another k-space line in another plane in the same cyclic fashion described in the first two methods. This interleaved acquisition can provide temperature map of all three orthogonal planes simultaneously. The advantages of this method include reduced imaging time and truly simultaneous acquisition of a temperature map in all three planes.

FIG. 3 shows an example of the pulse sequence diagram for the method of interleaved 3-plane acquisition. The pulse sequence diagram shows the alternating slice select/phase encode/readout direction between the three axes.

Software has also been developed to implement the method of the present invention. The image acquisition sequence follows as such:
A(1),B(1),C(1),A(2),B(2),C(2) . . . ,A(25), B(25),C(25) . . . ,A(256), B(256),C(256)
   in which P(i) denotes the i'th k-space line in plane P. The reconstruction software then sorts through these k-space lines to form complete data sets for each individual plane before Fast Fourier Transform (FFT). In the event of radial sampling, also called the projection reconstruction method of the k-space, the reconstruction sorts through these k-space lines before regrinding to form complete data sets for each plane. Lastly, the software reconstructs the phase difference images.

The inventor carried out a series of phantom experiments to demonstrate the feasibility of this technique. The experiments were performed using a Signa 1.5 scanner with a BRM gradient system. A head coil was used for the image acquisition. The following parameters were used during image acquisition:

| | | | |
|---|---|---|---|
| TR | 11.9 msec | Flip Angle | 30 |
| TE | 3.7 msec | Total Imaging Time | 9.87 sec |
| Slice Thickness | 3 mm | Field of View | 20.0 cm |
| Receiver Band Width | 31.25 kHz | Acquisition Matrix | 256 × 256 |

A number of small Agarose phantoms were made with T1 shortening Gd-DTPA contrast agent mixed. The contrast agent improves the signal to noise ratio and thus the sensitivity of the MR imaging machine to temperature variation. The phantoms were placed in a holder and located on three orthogonal axes 301, 302, 303 as shown in FIG. 4. First, a set of mask images was taken as the reference for later phase subtraction. The phantom at the center 311 was removed and heated before it was put back in the holder. The temperature of the phantom was raised to about 70° C. During the next 25 minutes images were taken every minute while the center phantom cooled towards room temperature. The other phantoms 310 were used as references for monitoring the system phase drift.

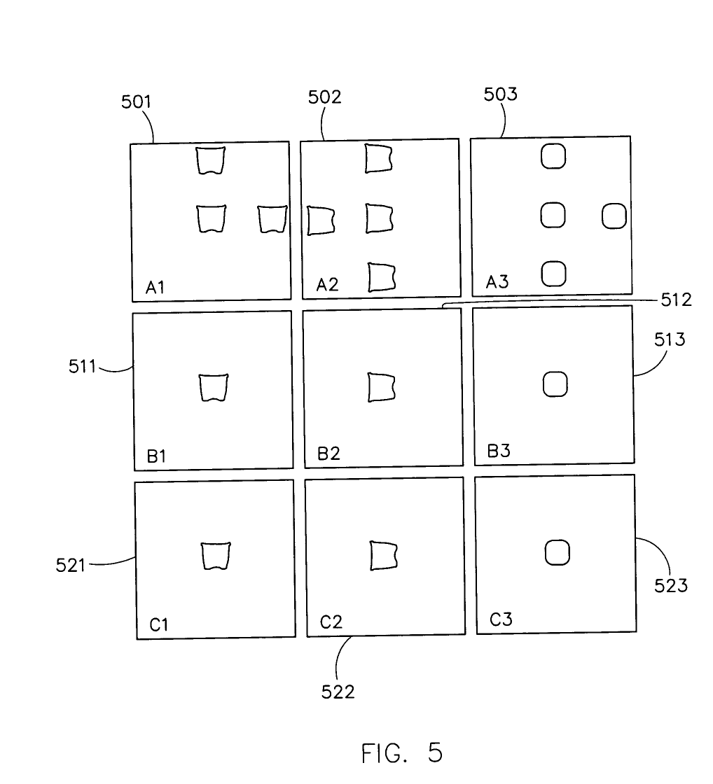

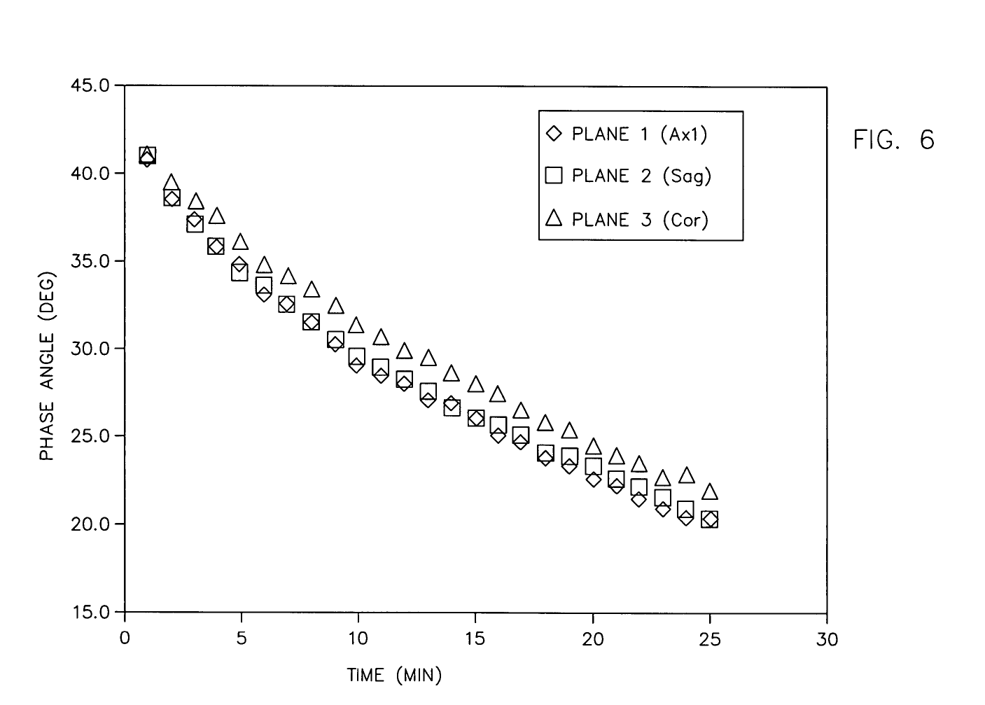

FIG. 5 shows the phase difference images and the magntitude image of three orthogonal planes 301, 302, 303 of the heated phantom both at the beginning of the cooling and at the end of the cooling period. The top row shows the magnitude image on three planes 501, 502, 503, A1 through A3. The middle row shows the phase difference images of the heated phantom as seen from three corresponding planes. The bottom row 511, 512, 513 shows the same images 521, 522, 523 as the middle row after 25 minutes of cooling.

Beneficially, the cross pattern of phantom images in the magnitude images is almost absent in the phase-different image. These patterns are caused by the interleaved acquisition method, which saturates the spins in the three orthogonal planes throughout the scan. It is an important advantage of the present invention to not have these effects in the phase different image as it would not provide any temperature information for the along these dark bands.

Analysis of the regions towards the center of the heated phantom was performed in all three planes. Mean values were then calculated. Similar analysis was then performed on each of the reference phantoms to determine the phase drift over the length of the experiment. It was found that a substantial amount of phase drift was experienced over the length of the experiment. The phase drift was then subtracted from the mean values of the temperature change. The results are shown graphically in FIG. 6. As is shown, change in the phase angle corresponds to a change in the temperature of the sample. The present invention provides a new and unique method observing the temperature change using an MR imaging system. It further provides for a method for using simultaneous three plane, two dimensional acquisition to represent three-dimensional temperature changes. It further provides for a method that is very efficient and effective in terms of acquisition and presentation. The method of the present invention accomplishes this by producing an image indicative of temperature change in a sample positioned in an MR imaging system wherein the MR imaging system acquires data from a plurality of k-space points. The method includes the steps of first performing an NMR pulse sequence to acquire phase reference images from the sample; wherein the NMR pulse sequence could be an RF-spoiled gradient echo pulse sequences. Second, a reference phase image is constructed from the sample. Third, a second NMR pulse sequence is used to acquire measurement NMR, wherein the NMR pulse could be an RF-spoiled gradient echo pulse sequence. The second NMR pulse sequence could further include the steps of: acquiring a first k-space line from a series of k-space points in a first k-space plane; acquiring a second k-space line from a series of k-space points in a second k-space plane; and acquiring a third k-space line from a series of k-space points in a third k-space plane. The method of the present invention could further include an image acquisition sequence that follows as such:
A(1), B(1),C(1),A(2),B(2),C(2) . . . ,A(25),B(25),C(25) . . . ,A(256),B(256),C(256)
   in which P(i) denotes the i'th k-space line in plane P. The fourth step in the method of the present invention includes measuring the signal phase shift. This fourth step could further include measuring the change in the resonance frequency of the water proton. The step wherein the signal phase shift is measured might further include the step of correlating the change in the resonance frequency of the water proton to a change in temperature. The last step of the method of the present invention is to produce a temperature map based on the phase differences. The method of the present invention also provides for periodically updating the reference phase image using measurement NMR data acquired during the scan. The method of the present invention could also include the step of repeating the steps of the image acquisition portion of the present invention so as to provide a plurality of additional temperature maps.

Additional advantages and modifications will readily occur to those skilled in the art. Therefore, the invention in its broader aspects is not limited to the specific details disclosed and described herein. Accordingly, various modifications may be made without departing from the spirit or scope of the general inventive concept as defined by the appended claims and their equivalents.

Parts List:

100 Console
102 Control Panel and Display
104 Screen
106 Image Processor Module
107 Computer System
108 CPU Module
111 Disk Storage/Memory
112 Tape Drive
113 Memory Module
115 High-speed Serial Link
116 Link
118 Back Plane
119 CPU Module
121 Pulse Generator Module
122 System Control
125 Serial Link
127 Gradient Amplifier
129 Physiological Acquisition Controller
133 Scan Room Interface Circuit
134 Patient Positioning System
139 Gradient Coil Assembly
140 Polarizing Magnet
141 Magnet Assembly
150 Transceiver Module
151 RF Amplifier
152 Whole Body RF Coil
152a Coil
152b Coil
153 Preamplifier
154 Transmit/Receive Switch
160 Memory module
201 RF Carrier Signal Output
202 Up Converter
204 Reference Signal
207 Receiver Attenuator
208 Down Converter
209 Analog-to-Digital Converter
210 Digital Detector and Signal Processor
230 Frequency Synthesizer
301 First Orthogonal Axis
302 Second Orthogonal Axis
303 Third Orthogonal Axis
310 Reference Phantoms
311 Center Phantom
501 First Plane Magnitude Image
502 Second Plane Magnitude Image
503 Third Plane Magnitude Image
511 First Plane Phase Difference Image
512 Second Plane Phase Difference Image
513 Third Plane Phase Difference Image
521 First Plane Phase Difference Image
522 Second Plane Phase Difference Image
523 Third Plane Phase Difference Image

1. A method for producing an image indicative of temperature change in a sample positioned in an MR imaging system, the steps comprising:
performing an NMR pulse sequence;
measuring the signal phase shift;
correlating the signal phase shift with a temperature change; and
constructing a temperature map.

2. The method of claim 1 wherein measuring the signal phase shift is further comprised of measuring the change in the resonance frequency of the water proton.

3. The method of claim 2 wherein measuring the signal phase shift is further comprised of correlating the change in the resonance frequency of the water proton to a change in temperature.

4. The method of claim 3 wherein the temperature map is method for constructing a temperature map further comprises:
acquiring a first k-space line in a first plane;
acquiring a second k-space line in a second plane; and
acquiring a third k-space line in a third plane.

5. The method of claim 4 wherein the image acquisition sequence follows as such:
A(1),B(1),C(1),A(2),B(2),C(2) . . . ,A(25),B(25),C(25) . . . ,A(256),B(256), C(256)
in which P(i) denotes the i'th k-space line in plane P.

6. The method of claim 5 wherein a scan is performed by repeating the steps of claim 5 to produce a corresponding plurality of additional temperature maps.

7. The method of claim 6 including the step of periodically updating the temperature map.

8. The method of claim 7 in which the NMR pulse sequences are RF-spoiled gradient echo pulse sequences.

9. The method of claim 8 in which a contrast agent is used in the sample.

10. The method of claim 9 wherein the contrast agent used is Gd-DPTA.

11. A method for producing an image indicative of temperature change in a sample positioned in an MR imaging system, the steps comprising:
performing an NMR pulse sequence to acquire phase reference images from the sample;
constructing a reference phase image from the sample;
performing an NMR pulse sequence to acquire measurement NMR data from the sample;
measuring the signal phase shift; and
producing a temperature map based on the difference phase differences.

12. The method of claim 11 wherein the measurement NMR data is comprised of a plurality of k-space points.

13. The method of claim 12 wherein the measurement of NMR data is comprises the steps of:
acquiring a first k-space line from a series of k-space points in a first k-space plane;
acquiring a second k-space line from a series of k-space points in a second k-space plane; and
acquiring a third k-space line from a series of k-space points in a third k-space plane.

14. The method of claim 13 wherein the image acquisition sequence follows as such:
A(1),B(1),C(1),A(2),B(2),C(2) . . . ,A(25),B(25),C(25) . . . ,A(256),B(256), C(256)
in which P(i) denotes the i'th k-space line in plane P.

15. The method of claim 14 which further includes periodically updating the reference phase image using measurement NMR data acquired during the scan.

16. The method of claim 15 which further includes the step of repeating the steps of claim 11 so as to provide a plurality of additional temperature maps.

17. The method of claim 16 wherein measuring the signal phase shift is further comprised of measuring the change in the resonance frequency of the water proton.

18. The method of claim 17 wherein measuring the signal phase shift is further comprised of correlating the change in the resonance frequency of the water proton to a change in temperature.

19. The method of claim 18 in which a contrast agent is used in the sample.

20. The method of claim 19 wherein the contrast agent used is Gd-DPTA.

21. The method of claim 20 in which the NMR pulse sequences are RF-spoiled gradient echo pulse sequences.

22. A method for producing an image indicative of temperature change in a sample positioned in an MR imaging system wherein the MR imaging system acquires data from a plurality of k-space points, the steps comprising:
performing an NMR pulse sequence to acquire phase reference images from the sample;
constructing a reference phase image from the sample;
performing an NMR pulse sequence to acquire measurement NMR data including the steps of;
(a) acquiring a first k-space line from a series of k-space points in a first k-space plane;
(b) acquiring a second k-space line from a series of k-space points in a second k-space plane; and
(c) acquiring a third k-space line from a series of k-space points in a third k-space plane.
measuring the signal phase shift; and
producing a temperature map based on the difference phase differences.

23. The method as recited in claim 22 which further includes periodically updating the reference phase image using measurement NMR data acquired during the scan.

24. The method as recited in claim 23 which further includes the step of repeating the steps of claim 11 so as to provide a plurality of additional temperature maps.

25. The method as recited in claim 24 wherein measuring the signal phase shift is further comprised of measuring the change in the resonance frequency of the water proton.

26. The method as recited in claim 25 wherein measuring the signal phase shift is further comprised of correlating the change in the resonance frequency of the water proton to a change in temperature.

27. The method as recited in claim 26 wherein the image acquisition sequence follows as such:
A(1),B(1),C(1),A(2),B(2),C(2) . . . ,A(25),B(25),C(25) . . . ,A(256),B(256), C(256)
in which P(i) denotes the i'th k-space line in plane P.

28. The method as recited in claim 27 in which the NMR pulse sequences are RF-spoiled gradient echo pulse sequences.
